(19) United States
(12) Patent Application Publication
(10) Pub. No.: US 2003/0224370 A1
(43) Pub. Date: Dec. 4, 2003

(54) Method and apparatus for recognizing molecular compounds

(76) Inventors: William Rassman, Los Angeles, CA (US); David Ralin, San Gabriel, CA (US); Feiming Zhou, Temple City, CA (US); Shubo Han, Alhambra, CA (US)

Correspondence Address: Marvin H. Kleinberg, KLEINBERG & LERNER, LLP, Suite 1080, 2049 Century Park East, Los Angeles, CA, 90067, US

(21) Appl. No.: 10/158,995

(22) Filed: May 31, 2002

(51) Int. Cl. C12Q 1/68; G01N 33/53; G01J 3/45; G01B 9/02

(52) U.S. Cl. 435/6; 435/7.1; 356/451

(57) ABSTRACT

A probe-target reaction is made more recognizable by the provision of a mass-enhancing and/or evanescent-field-perturbing amplifier element which reacts uniquely with and binds to the probe-target pair to provide increased mass. Where the probe-target pair is hybridized dsDNA, a suitable mass-enhancing amplifier is anti-double stranded DNA mouse IgM. In examples with sufficient sequence pairs in the probe-target combination, a sequence-specific minor-groove-binding polyamide can be used that carries biotin which can be amplified by streptavidin in a suitable carrier. In a preferred embodiment, a plurality of probes are immobilized at the sites of a microarray, each probe being specific to a different target. Optics utilizing total internal reflection are described for observing perturbation of the evanescent field.

### BACKGROUND OF THE INVENTION

1. Field of the Invention

The present invention relates to methods and apparatus for detecting, qualitatively and quantitatively, molecular interaction.

2. Description of the Related Art

To help describe the prior art and the present invention, it is deemed useful to provide a glossary of terms that will be used herein. A “probe” is intended to mean a known entity which may either be immobilized or free floating. A “target” or “ligand” is an “unknown” entity which has a known, specific complementary or reactive relationship with a specific probe. Probe-target combinations may be referred to as “complexes” or “pairs”.

A “primary amplifier” or “mass enhancing unit” is a “bulky” entity which has an interaction or bonding with a pair, or with a linking element that bonds to the pair. A “secondary amplifier” interacts with the primary amplifier or with the combination of primary amplifier-probe-target pair complex to form a new combination. A “tertiary amplifier” is any successive mass enhancer which can interact with a primary amplifier-probe-target pair complex or amplifier.

“Bulk factor” is the ratio of the molecular weight of the amplifier to that of the probe-target pair. However, bulk or mass is not always the primary parameter in enhancing recognition of a probe-target pair. Other parameters, such as optical or electrical properties, may be important to a detection device.

Researchers working with probe-target reactions to identify an “unknown” analyte have generally relied upon marking or tagging “target” compounds so that a reaction between probe and target molecules could be detected and signaled. Conventional techniques included the use of a linking compound to which fluorescent, radioactive, or “colored” tags or markers could bind or attach. Techniques were developed to detect and signal these markers. As a result, these tests would always require the extra steps involving tagging or marking steps.

In prior, copending applications of the common assignee, it has been taught that molecular reactions which take place in the evanescent field of a total internal reflecting (TIR) surface, can be detected utilizing a polarized beam and additional optical elements which can respond to changes in the evanescent field to provide a distinctive display, recognizable by an optical detector such as a CCD camera chip. Other detection devices include spectrometers, gravimetric devices, size excluding devices and charge-mass-ratio separating devices.

The prior applications, the teachings of which are incorporated herein, include U.S. application Ser. No. 09/614,503, filed Jul. 7, 2000, application Ser. No. 09/838,700, filed Apr. 19, 2001, and application Ser. No. 10/046,620, filed Dec. 12, 2002.

In these applications, a reaction takes place on a surface in the evanescent field of a total internal reflecting (TIR) surface. A polarized beam of light is directed at the surface and totally reflected therefrom. The presence of matter in the evanescent field modifies the polarization properties of the reflected beam,

Before, during and after the reaction, the resultant beam is directed to a detector, for example, a CCD chip/camera, which can visualize the entire surface of interest. The reacted area then provides a feature which differs from the non-reacted areas. The specific location of the feature identifies the reaction which produced the feature.

Detecting the unique feature can present a challenge if the reagents, and the reacting and non-reacting areas are sufficiently small so as to require extremely sensitive detection systems. Generally, the prior art has relied upon tagging or marking to enable the identification of a reaction. In the applications commonly assigned to the assignee of the present invention, the marking or tagging step was unnecessary. However, highly sensitive techniques were required to detect the reaction and identify the reactants.

### SUMMARY OF THE INVENTION

According to the present invention, a complex product which can be the result of any reaction (e.g., an antibody in the case of bioactive materials), is used to develop, enhance or “amplify” a probe-target reaction so that the resultant compound can be detected with less sensitive equipment or the detection limit of sensitive equipment may be effectively lowered so that smaller quantities of the target may be detected.

For example, it is known that there are “probe” molecules that have an exclusive or specific affinity for a “target” molecule resulting in the formation of a probe-target pair. Further, there are “amplifier” molecules or compounds that have a specific attraction or affinity for a class of probe-target pairs, or for a particular subset of a class of probe-target pairs.

Nucleic acid probes can react with complementary RNA or DNA sequences to hybridize, forming a double stranded sequence. Once hybridization has taken place, an antibody specific to lengths of double stranded nucleic acid material is added and incubated. Generally, the antibody will have a molecular weight and bulk many times that of the probe and target, resulting in an enlarged and enhanced probe-target combination that is more readily detected, even with less sensitive optical or other systems.

In the patent to Carter et al, Pat. No. 4,508,832, the use of ellipsometry was described in conjunction with a bioassay method. As described by Carter et al, bioassays or immunoassays are based on the reaction of a bioactive substance (or “antigen”) with a specific complex conjugate of that bioactive substance (or “antibody”). It is believed that the present invention can be extremely useful in the performance of bioassays or immunoassays. Furthermore, U.S. Pat. No. 6,228,578 to Ipraim et al., the entirety of which is incorporated herein by reference, describes use of an immunoglobulin with a specific affinity for DNA probe/RNA target hybrids to recognize the hybrids. The probe-target-amplifier is detected only by further processing resulting in chemiluminescence or by use of a fluorescent secondary immunoglobulin which specifically binds to the hybrid-specific immunoglobulin.

In the present invention, detection may occur at any point during or after hybridization, with increasing sensitivity afforded by mass amplification. In the particular experimental embodiments employed to demonstrate the method of the present invention, single stranded DNA (“ssDNA”) is the probe. A surface can be prepared with a matrix of probes affixed thereto, each with a different sequence. The x-y location in the matrix is known for each probe element. The unknown target compound is prepared as ssDNA and is reacted with the probe material. If the probe and target have complementary sequences, there will be a reaction at the site and the two single strand DNA samples will hybridize into a double stranded DNA (“dsDNA”) chain. A reaction at any particular x-y location uniquely identifies the unknown or “target” in the test.

Primary amplifying antibodies, whose antigen is dsDNA, are now introduced to the matrix surface. The amplifying antibodies are selected for their specificity for dsDNA as determined by the structure of their variable region. The amplifying antibodies are generally an order of magnitude more massive than the hybridized pair. All antibodies of a particular class, i.e. IgA, IgM, IgG, etc., have different molecular structures and mass and all are relatively bulky. At the site where hybridization has occurred, the amplifying antibodies will bind to the dsDNA. Under certain conditions, the amplifying antibodies will aggregate or agglomerate, nucleating upon the antibody bound to the probe-target pair, resulting in a structure of massive size and bulk. Alternatively, the active Fab fragment or F(ab′)₂ fragments may be separated from the immunoglobulin by papain or pepsin cleavage, respectively, providing a smaller, sterically and kinetically favorable recognition unit. In this case, biotinylation of the antibody fragments would provide for linkage to avidin or avidin-containing bulky compounds.

Alternatively, secondary amplifying antibodies, specific for the primary amplifying antibodies, could be added which will bind to the primary antibodies to create, at a particular x-y location, a larger object, detectable with less sensitive equipment, such as relatively insensitive optics and easily recognized by using a camera which can visualize the entire matrix. Such an aggregation or agglomeration could be detected by an atomic force microscope, among other surface techniques, which can determine height profile changes.

In an alternative application, non-human IgG and human immunoglobulins are the probe-target pair, as a means to detect antibodies in human serum, thereby determining if a person has been exposed to a particular antigen. If IgM that is specific for the non-human anti-human-immunoglobulin complex, or even specific just for the human immunoglobulin, is used as the primary amplifying antibody, the bulk of the pair can be increased two-and-a half fold. Yet other probe-target pairs can be identified, with the amplifying antibodies that can be used with such pairs.

In additional alternative embodiments, yet other, secondary amplifying antibodies can be employed, with or without markers, that are reactive with either the hybridized pair or with the primary amplifying antibodies to increase the detectability of the reacted pair through techniques which utilize separating processes that are based upon mass differentiation.

The present invention can be utilized with other analytes of interest. In such other embodiments, results can be observed with electron or conventional microscopes, colorimetry, gravimetric analysis, chromatography and spectroscopy. If it is desired to use tags or markers, yet other techniques of the prior art could be employed at much lower levels of sensitivity.

Accordingly, it is an object of invention to provide a more easily identified target-probe combination which can employ less sensitive techniques for analyzing results.

It is another object of invention to provide a more easily identified target-probe combination which can exhibit lower levels of detection for sensitive detecting equipment. It is a further object of invention to amplify the result of a probe-target interaction permitting the use of less sensitive detection schemes.

It is a further object of invention to amplify the result of a probe-target interaction thereby lowering the limits of detection.

It is yet another object of the invention to utilize compounds that are specific for molecular probe-target complexes to “amplify” the signal response to the probe-target reaction.

It is still another object of invention utilize secondary amplifying compounds that can bind to the primary amplifying compounds that are bound to the specific probe-target reactions, or, ideally, the probe-target-primary-amplifier complex.

The novel features which are characteristic of the invention, both as to structure and method of operation thereof, together with further objects and advantages thereof, will be understood from the following description, considered in connection with the accompanying drawings, in which the preferred embodiment of the invention is illustrated by way of example. It is to be expressly understood, however, that the drawings are for the purpose of illustration and description only, and they are not intended as a definition of the limits of the invention.

### BRIEF DESCRIPTION OF THE DRAWINGS

For a further understanding of the objects and advantages of the present invention, reference should be had to the following detailed description, taken in conjunction with the accompanying drawing, in which like parts are given like reference numbers and wherein:

FIG. 1 is a schematic representation of a prior art detection scheme relying on a radioactive or fluorescent label;

FIG. 2 is a schematic representation of an exemplary detection technique in accordance with the present invention;

FIG. 3 is a chart correlating size with the various stages taught with reference to an exemplary embodiment of a method and apparatus in accordance with the present invention;

FIGS. 4 AND 5 depict the optical observation via total internal reflection as an aspect of an exemplary embodiment of a method and apparatus in accordance with the present invention;

FIG. 6 is a chart associating layer thickness with various probe-target pairs as an aspect of an exemplary embodiment of a method and apparatus in accordance with the present invention;

FIG. 7 is a table associating layer thickness with various probe-target pairs as an aspect of an exemplary embodiment of a method and apparatus in accordance with the present invention; and

FIG. 8 is a table summarizing exemplary approaches to using the method and apparatus in accordance with the present invention.

### DESCRIPTION OF THE PREFERRED EMBODIMENTS

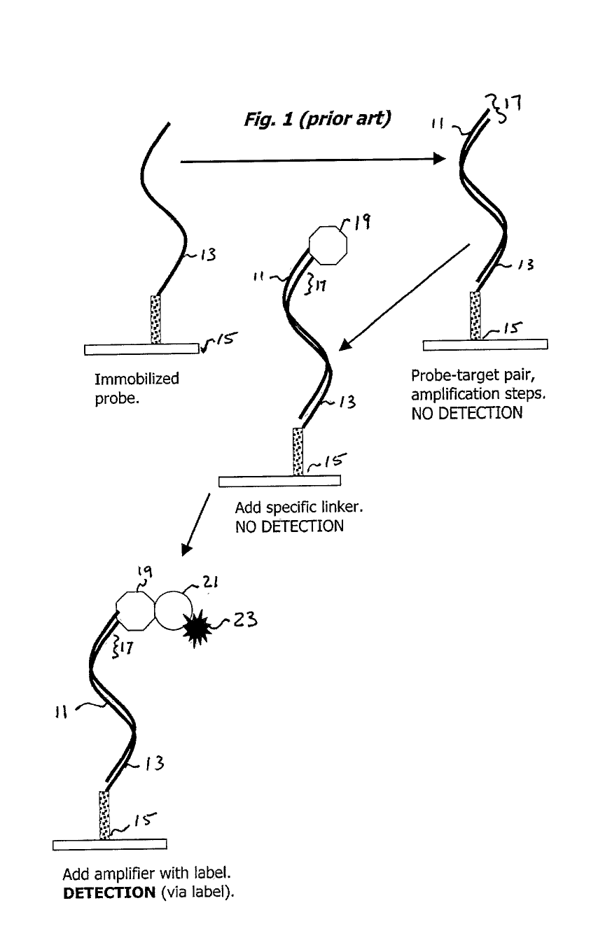

FIG. 1, shows the prevailing prior art sequence in which a target 11 hybridizes with the probe 13 which has been bound to a substrate 15 to form a probe-target pair 17 whose identity is to be ascertained is made detectable. It has been a common practice in the prior art to add a specific linker 19 to which an amplifier 21 which has a tag or label 23 can bind. The presence of the tag or label 23 is usually the result of a separate processing step which may include the addition of the linking ligand upon the target. It is the presence of the tag or label, however, that makes the probe target pair 17 detectable. Equipment which is sensitive to the tag or label 23 allows the target to be uniquely identified.

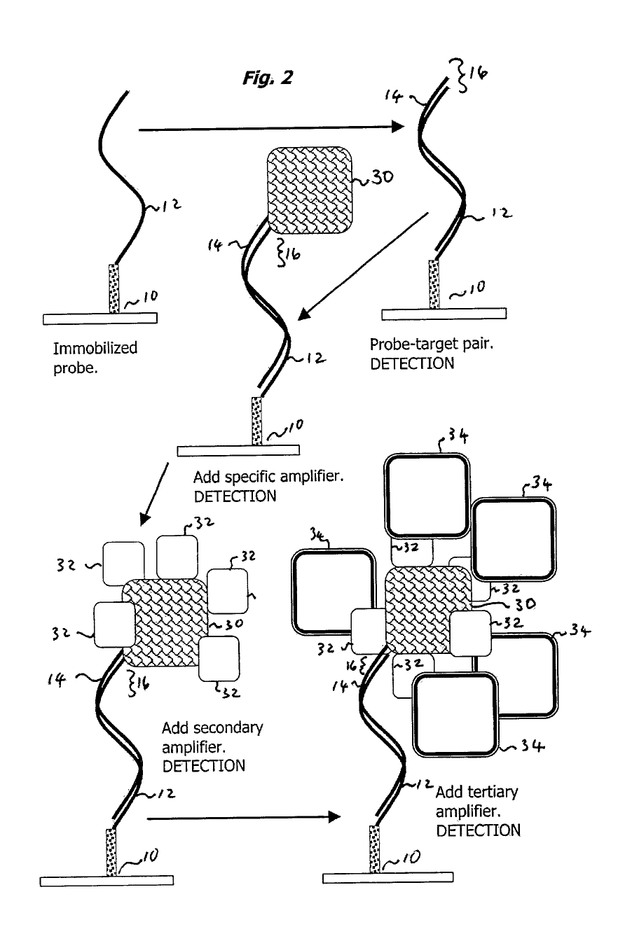

FIG. 2 shows for an exemplary embodiment of the method and apparatus in accordance with the present invention a probe element 12 which can be a ssDNA fragment bound to a substrate 10. In a preferred embodiment, the substrate 10 may be a glass slide.

With continued reference to FIG. 2 is shown, in a series of steps, the combination of the probe ssDNA 12 with the target ssDNA 14, hybridizing to form a dsDNA pair 16. For such hybridized dsDNA pairs 16, there exist antibodies or primary amplifying bodies 30. These primary amplifying bodies 30 can only bind to the hybridized dsDNA pairs 16 but not to the ssDNA probe 12 or target elements 14.

It is thus possible to aggregate or agglomerate a rather massive structure at the site of the probe-target pair so that there is a single, unique, massive structure in the matrix that can be more readily detectable by less sensitive detectors including, but not limited to, optical, electrical, topographic, gravimetric, or other mass discriminating techniques and others.

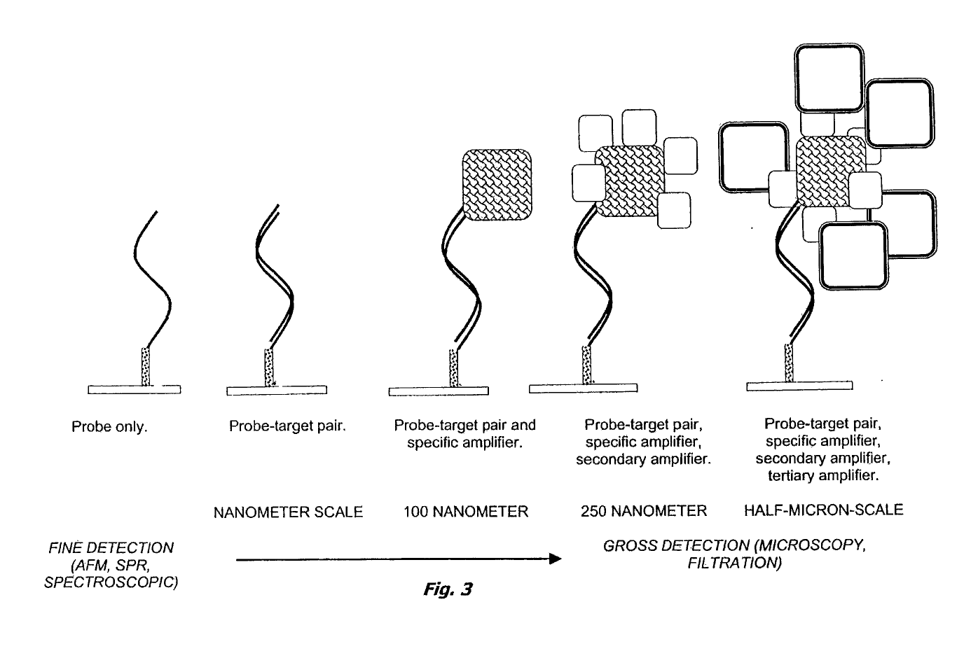

FIG. 3 is a progression showing relative size of the various resultant combinations starting with the substrate 10 with an immobilized probe 12. As the legend indicates, the probe-target pair 16 can be measured in fractions of nanometers of film thickness on the surface of the substrate 10. With the addition of a primary amplifying body 30, the scale increases to the 30 nanometer range, detectable by sensitive systems such as atomic force microscopes, or the techniques of the copending applications of the common assignee. With these values, other “fine” detection systems, such as spectrometers or gravimetric devices, can find evidence of the probe-target reaction.

The techniques of the present invention are useful with any probe-target pair for which specific amplifier compounds can be identified. It is believed that the technique can be applied in any situation involving an unknown material or target at the molecular stage which can react with a probe of comparable size. Amplifying materials can be identified which can bind to the probe-target combination.

### EXAMPLE1

### Oligonucleotide Probe and Target; IgM Primary Amplifying Body

One area in which the present invention is useful is with the dry state surface scanning detection of an oligonucleotide probe-target pair on an aldehyde derivatized glass substrate. The process entails the following steps:

Clean a Superaldehyde (Telechem International) slide using a nitrogen stream;

Using a Tris-EDTA-NaCl buffer (“TE-NaCl buffer”), spot a 30 mer oligonucleotide modified with C6 amino at 3′ end to the slide surface to act as a probe.

Dry the probe for 12 hours at room temperature (25° C.) and <30% relative humidity.

Rinse the substrate twice with vigorous agitation in 0.2% SDS (sodium dodecyl sulfate) for two minutes to remove unbound oligonucleotides (DNA).

Rinse the substrate once with vigorous agitation in double distilled water (ddH2O) for two minutes.

Dry the substrate with a nitrogen stream.

Prepare a fresh sodium borohydride solution by dissolving 1.5 g NaBH₄ in 450 ml. Phosphate buffered saline (PBS). Add 133 ml 10% ethanol.

Treat the substrate with this solution for five minutes at room temperature to reduce free aldehydes.

Rinse the substrate twice in 0.2% SDS for one minute at room temperature.

Rinse the substrate once in ddH₂O for one minute at room temperature.

Dry the prepared substrate in a nitrogen stream.

Soak the substrate with the complementary oligonucleotide target in TE-NaCl (Tris-EDTA, 1M NaCl) buffer for 12 hours at room temperature.

Dry the prepared substrate with a nitrogen stream.

Soak the substrate with anti-dsDNA amplifying antibody solution in PBS buffer for one hour at room temperature.

Dry the prepared substrate with a nitrogen stream.

The prepared substrate can then be “read” by an atomic force microscope which will detect the hybridized pair and the associated amplifier.

Polystyrene microspheres may be obtained from Bangs Laboratories, Inc. (9025 Technology Drive, Fishers, Ind. 46038-2886; www.bangslabs.com) in derivatized form in sizes from 25 nm to 1000 nm, the IgG being supplied by the customer. The IgG is attached via the Fc portion, leaving the variable region exposed and available for coupling to antigens. 100 nm spheres are a preferable first choice. The microspheres may be dyed to absorb the wavelength of interest if an optical method of probing is desired. The microspheres may be coupled to goat anti-mouse-IgM serve as a secondary amplifier for a primary amplifying antibody. A preferred method is as follows:

Prepare the hybridized surface and add the IgM as set forth in EXAMPLE 1.

Prepare a diluted microsphere solution by diluting the stock solution 10× in PBS.

Incubate the surface with 10 microliters of the diluted solution for 30 minutes

Rinse with PBS

Rinse with dH2O (optional, if using a dry reading method)

Observations: At least 10× signal enhancement.

If a SEQUENCE SEEKER™ commercial sequence-specific polyamide product is used to bind to a double-stranded probe-target pair, streptavidin-coated polystyrene microspheres are preferred (dyed or un-dyed, as needed) (SEQUENCE SEEKER™, Prolinx, Inc., Bothell, Wash.; www.prolinx.com).

Prepare the hybridized surface

Dilute dry SEQUENCE SEEKER™ in a mass equivalent volume of absolute ethanol.

Dilute the SEQUENCE SEEKER™ to 0.1% mass/volume in TE buffer. Sonication helps to dissolve.

Incubate the surface with 10 microliters of the solution for 30 minutes

Rinse the surface with TE buffer to remove unbound SEQUENCE SEEKER™.

Prepare a diluted microsphere solution by diluting the stock solution 10× in PBS.

Incubate the surface with 10 microliters of the diluted solution for 30 minutes.

Rinse with TE buffer.

Observations: At least 10× signal enhancement.

(Optional, if using a dry reading method) Rinse with dH2O.

In addition to the use of antibodies, which can bind to hybridized or dsDNA, there are commercially available polyamides which are commercially known as SEQUENCE SEEKER™ (Prolinx, Inc., Bothell, Wash.; www.prolinx.com) and which can be conjugated with specific compounds, for example biotin. The SEQUENCE SEEKER™ is drawn to strands of hybridized DNA and if the sequence contains the right sequence of five base pairs, the polyamide will bind itself to the minor groove of the dsDNA.

In this example, the sequence seeker is not the amplifier, but serves as a “recognition unit” similar to the specific peptide structure in the variable region of the antibody, which is responsible for specificity. A biotin is bound to the sequence seeker to act as a linking compound with streptavidin or streptavidin-coupled compounds, which may serve as the mass amplifier. A preferred method is as follows:

Dilute dry biotinylated SEQUENCE SEEKER™ in a mass equivalent volume of absolute ethanol.

Dilute the SEQUENCE SEEKER™ to 0.1% mass/volume in TE buffer. Sonication helps to dissolve.

Incubate the surface with 10 microliters of the solution for 30 minutes.

Rinse the surface with TE buffer to remove unbound SEQUENCE SEEKER™.

If using streptavidin as the amplifier:

Prepare a 100 micromolar solution of streptavidin in TE buffer.

Incubate the surface with 10 microliters of the diluted solution for 10 minutes.

Rinse with TE buffer.

(Optional, if using a dry reading method) Rinse with dH2O

Observations: Signal increasing by ratio stated on chart, or up to surface saturation.

If using streptavidin-coupled microspheres:

Prepare a diluted microsphere (streptavidin microspheres, Bangs Labs) solution by diluting the stock solution 10× in PBS.

Incubate the surface with 10 microliters of the diluted solution for 30 minutes.

Rinse with TE buffer.

(Optional, if using a dry reading method) Rinse with dH2O.

Observations: Signal should increase 50-fold, up to the surface saturation point of the microspheres.

Some primary amplifying bodies 30 can bind or agglomerate to primary amplifying bodies that have been bound to a probe-target pair.

With reference again to FIGS. 2 and 3, there are also secondary amplifying bodies 32 which can bind to the primary amplifying bodies 30 that are bound to a hybridized probe-target pair 16. It is then possible to aggregate or agglomerate a rather massive structure at the site of the probe-target reaction so that there is a single, unique, massive structure in the matrix that can be more readily detectable by less sensitive detectors including, but not limited to, optical, gravimetric, topographical or other mass discriminating techniques and others.

Adding the secondary amplifying bodies 32 increases the size by a factor of approximately 2.5. A preferred method is as follows:

Dilute stock antibody solution 10× in PBS to make 10 microliters.

Incubate previously prepared probe-target-IgM substrate with solution for 30 minutes.

Rinse with PBS

(Optional, if using a dry reading method) Rinse with dH2O.

Observation: signal approximately doubles, if four IgG attach to each IgM at the surface.

Streptavidin is a protein which has an affinity for biotin (Kassoc=10¹⁵) and one or more molecules of streptavidin can bind to the biotin that is carried by the SEQUENCE SEEKER™. The streptavidin itself is more massive (68 kilodaltons) than the dsDNA probe-target pair, providing a primary mass amplifying effect.

Examples of a non immunoglobulin antigens as a targets can include protein arrays having importance to agricultural industry in certifying that processed foods are free of contaminating allergens, GMOs. Police and national security agencies may wish to use the present invention to improve existing arrays that are used to detect explosives such as TNT.

Preferably, the probe element 12 is part of a matrix of other, different probes (or ssDNA fragments). Known matrix arrangements are capable of providing a match to an unknown target probe or ssDNA, the identity of which is to be found by a test.

A procedure for this exemplary embodiment includes the steps recited for EXAMPLE 1 above for oligo probes, except that there are numerous different probes placed in a multi-well plate (for example, a 396-well plate). The plate is placed in a spotting robot, which prints the oligo probes on the slide in an arrayed pattern determined by the software and experimenter.

The substrate can have total internal reflection (TIR) as taught in the prior patent applications of the common assignee, placing the probe element in the evanescent field.

With reference again to FIGS. 2 and 3, there are also tertiary amplifiers 34 which bind to the secondary amplifying bodies 32, creating even more massive combinations at the site of the probe-target reaction, lowering the threshold of detection in many detecting schemes.

The ability to bind a tertiary amplifier 34 to the site would provide an additional twenty-fold size increase from the initial probe-target thickness value. The addition of secondary and tertiary amplifying bodies or amplifiers permits the use of “gross” detection systems such as microscopy, filtration or other size exclusion techniques and any mass based separation system.

Streptavidin is readily available coupled to a variety of materials, such as polystyrene, polyaniline, or metallic microspheres, which are even more massive. Additionally, streptavidin has four binding sites for biotin, allowing biotinylated antibodies or biotinylated microspheres to be attached, serving as the secondary amplifying body, and the streptavidin itself also serves as the specific target for antibodies, which in that case would be the secondary amplifying body.

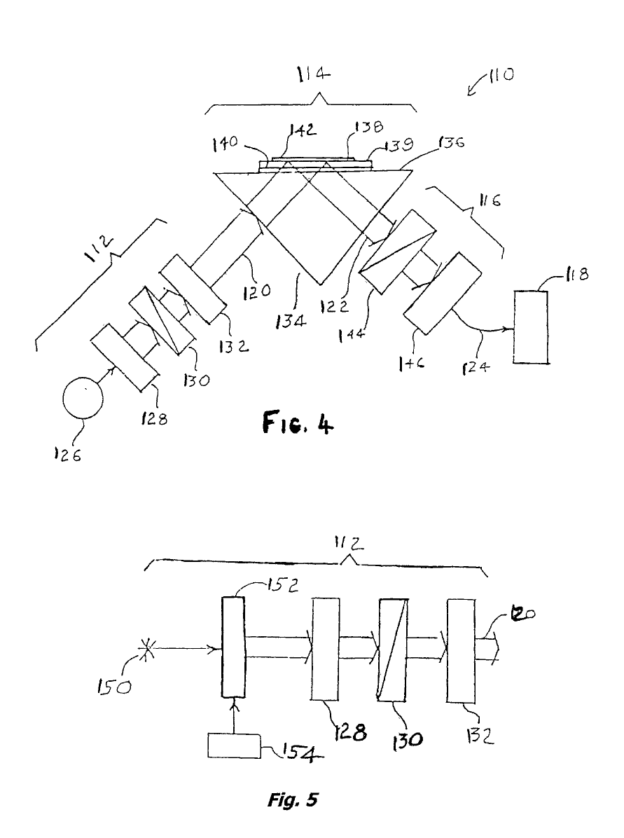

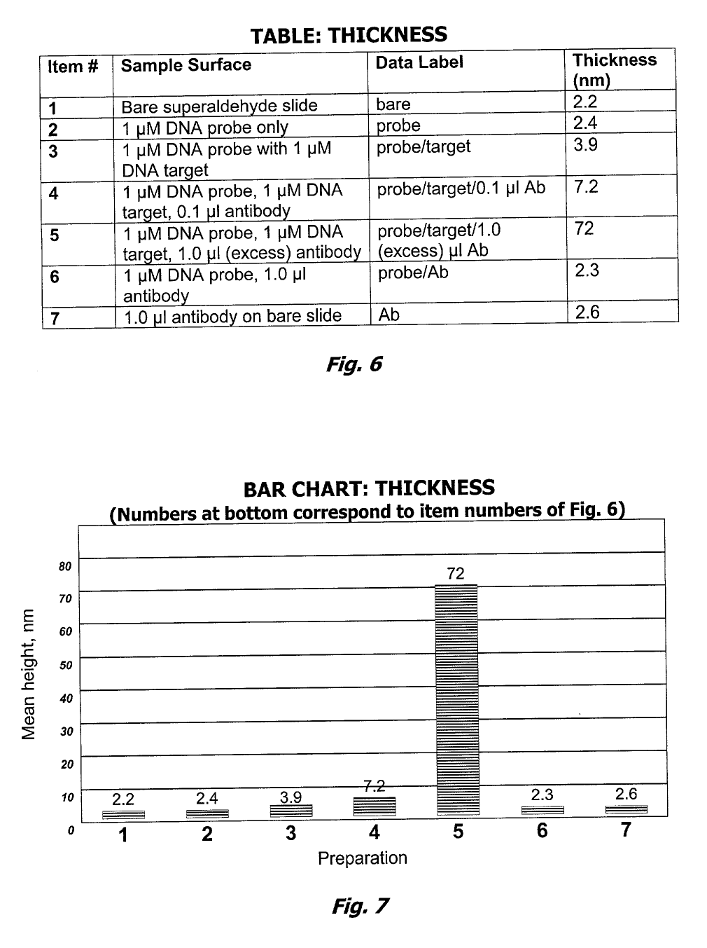

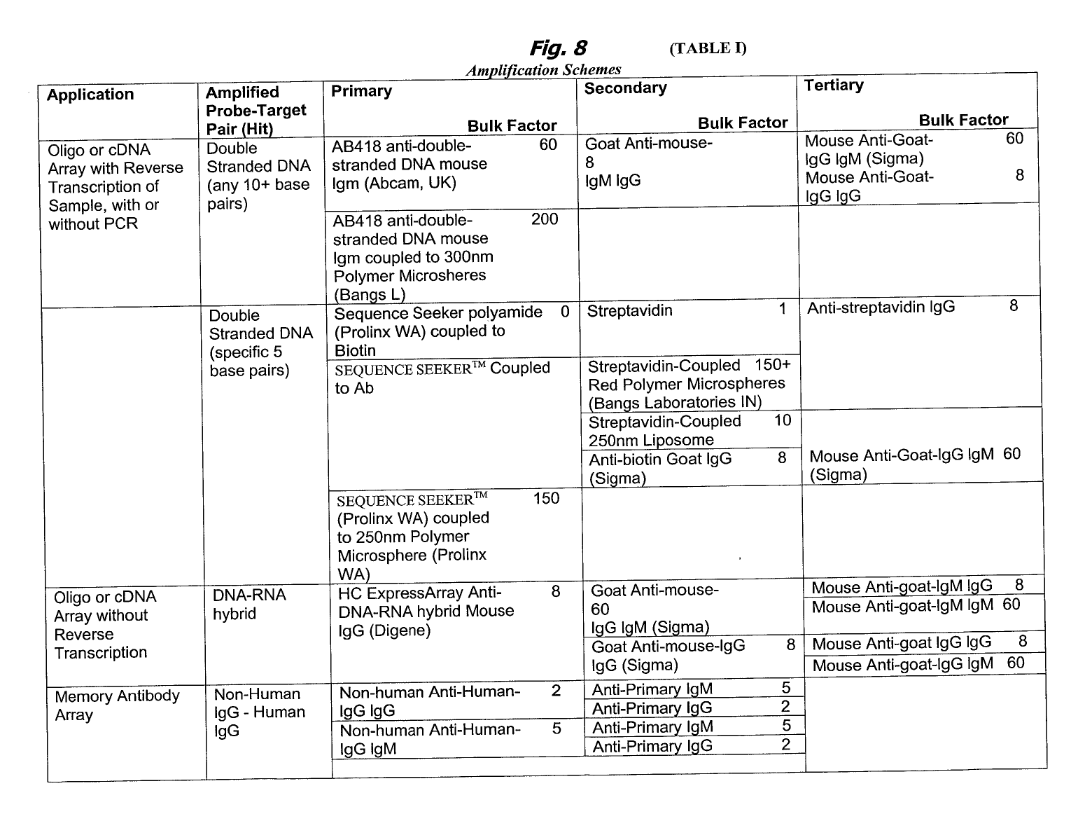

FIG. 5 is a bar chart showing relative heights expressed in nanometers (nm). As seen in the chart, the bare aldehyde slide surface has a height of 2.2 nm. With attachment of a 1 μM DNA probe, the height increases to 2.4 nm. The probe-target pair of the 1 μM DNA probe and 1 μM DNA target has a height of 3.9 nm. With the addition of a primary amplifier, 0.1 μl of antibody, the height increases to 7.2 nm. However, if excess antibody is added as the primary amplifier, the height is increased tenfold to 72 nm.

For confirmation of the experiment, the height of a 1 μM DNA probe with 1 μl of antibody is only 2.3 nm and the height of the antibody alone on a bare slide is only 2.6 nm. From this chart, it is clear that the hybridized probe-target pair in the presence of sufficient primary amplifying material will exhibit a significant change in height which is readily detectable.

The chart of TABLE I (see FIG. 8, SHEET 6) sets forth suitable probe-target combinations and the primary, secondary and, in some cases, tertiary amplifiers that are suited to each combination.

It should also be noted that, as an alternative to the SEQUENCE SEEKER™, one may also specifically identify a particular double stranded nucleic acid sequence by means of a type of nucleic acid analog known as a locked nucleic acid (“(LNA™”, Proligo LLC, www.proligo.com; subsidiary of Degussa AG, www.degussa.com).

FIG. 4 shows an exemplary embodiment of the present invention in which a total internal reflection (TIR) is used to observe the result of amplification. A polarized light source assembly 112 has a light source 126, a beam forming member 128 (if the nature of the light source is such as to make beam forming useful or necessary), a polarizer 130 and an optical retarder 132. The total internal light reflection assembly 114 has an optical element 134 which has an optical surface 136. Also shown is a specimen slide 138 on the optical surface 136, and between them an index matching substance 140. Because of the index matching a total internal reflection surface (TIR surface) is defined as the upper surface 139 of the specimen slide 138. A specimen 142 is on the TIR surface 139 of the slide 138.

The optical element 134 is a prism configured along with the index matched slide 138 in relationship to the incoming light beam 120 and the exiting light beam 122 such that the beam reflects only a single time at the TIR surface 139 and then exits the prism. If the specimen is placed directly on the optical surface 136, then the optical surface 136 would be the TIR surface. But this is not the usual application as the specimen (such as a biochip) is usually prepared more conveniently on a specimen slide 138 and placed in the apparatus.

In any event, however constructed, there is an optical structure having a TIR surface and the beam reflects only a single time at the TIR surface between entering and leaving the optical structure. In other words, there is a TIR surface in optical contact with the specimen, such that the evanescent field associated with the total internal reflection interacts with the specimen, and there is only a single reflection at that TIR surface.

The post-reflection detector assembly 116 has a polarizer 144 and a two-dimensional array detector 146, preferably a camera of the CCD type. The processor 118 is a specially programmed computer and output means for processing the image into a representation of film thickness variations spatially resolved over the area imaged. A image is acquired by detecting changes spatially distributed in the local polarization state in the beam's cross-section caused by the total internal reflection. This provides information about the presence and composition in the array of substances on the substrate surface for each resolvable point on the surface. Different polarization state changes are included in the cross-section of the reflected beam indicative of the substances on the specimen in the location in the specimen array corresponding to a position in the detector. The processor 118 receives the data as an electrical signal 124 and characterizes the change of polarization state spatially over the two-dimensional array. In the processor 118, the analysis and processing is done in one embodiment by comparing the known polarization state of the incoming light from the light processing assembly 112 with the changed polarization state of the reflected light 122, spatially resolved two-dimensionally within the beam which provides a map of spatially distributed points of spots in the specimen array. The polarization shift is then analyzed by the processor 118 to provide information of the presence and properties of elements in the chemical specimen. Other known techniques, such as null processing or phase modulation can be used to determine the change in polarization state.

Alternatively, the light source ember 126 may be a light emitting diode (LED), a superluminescent diode (SLD), an incandescent light source, or a laser. If an LED or SLD is used, the set-up shown in FIG. 4 is appropriate, where the beam forming member 128 is a collimator. if an incandescent light source is used, an optical filter is also used.

In one embodiment, the light source 126 for the apparatus is a quasi-monochromatic light sources of moderate bandwidth. In accordance with the invention, the light source 126 is preferably an LED of moderate bandwidth. Preferably the bandwidth is a full width half maximum wavelength in the range of about 10 nanometers to 50 nanometers, and more preferably a full width half maximum wavelength in the range of about 30 nanometers to 50 nanometers.

With reference to the optical retarder 132 as shown in FIG. 4, in an alternative embodiment, the optical retarder could be placed instead in the exiting beam path 122 before the polarizer 144. With reference to FIG. 5, an alternative embodiment is shown. When the light source is a laser 150, a moving diffuser 152 is adapted to produce speckle offsetting fluctuation of the minima and maxima in the speckle pattern caused by the laser. The moving diffuser 152 is attached to a mechanical actuator 154 which is preferably a moter and servoapparatus for providing the speckle offsetting fluctuations. The beam 120 then proceeds through the beam forming element 128, the polarizer 130 and the optical retarder 132, exiting the light source assembly 120.

For detection by electrochemical or permittivity instruments, metal nanospheres coupled to recognition units, primary amplifiers, or secondary amplifiers could be used. In one developed methodology, streptavidin-coupled gold nanospheres which are in turn coated with colloidal silver are used to amplify biotinylated biomolecules.

If biotinylated DNA, RNA, or protein targets are used, enhancement with streptavidin-gold nanoparticles, with or without silver colloid enhancement, will amplify signals when probed by ellipsometry, reflectometry, evanescent techniques, light microscopy, high resolution scanning, scanning electrochemical probe microscopy, and AFM.

A general procedure, adaptable to the particular probe-target system by those skilled in the art, is as follows:

Incubate a hybridized array with streptavidin-Nanogold® (Nanoprobes, Inc., Yaphank, N.Y.; www.nanoprobes.com) diluted 1:200 to 1:500 in PBS containing 1% BSA at room temperature for 60 min.

Wash in 3 changes of PBS containing 0.1% fish gelatin and 0.1% Tween-20 for 5 min each.

Repeatedly wash in distilled water for at least 10 min altogether, the last 2 rinses in ultrapure water (EM-grade). Prepare solution A and B:

Solution A: Dissolve 80 mg silver acetate (code 85140; Fluka, Buchs, Switzerland) in 40 mL of glass double-distilled water. (Silver acetate crystals can be dissolved by continuous stirring within about 15 min.)

Solution B: Dissolve 200 mg hydroquinone in 40 mL citrate buffer.

Just before use, mix solution A with solution B.

Incubate the gold-septavidin enhanced array with the enhancement solution for 30 minutes.

Wash three times with distilled water.

Probe the array with the preferred detection method. In-situ processing and probing, using a sealed flow cell apparatus and an amenable detection system, will allow probing during hybridization and processing, permitting dynamic measurements while reducing handling artifacts.

Thus there has been shown and described a method and materials for detecting probe-target combinations at relatively lower levels of sensitivity. A mass amplifier is added to a probe-target pair (with or without a linking element). The presence of the mass amplifier, without other process steps, permits detection of the probe-target pair. Additional mass amplifiers, such as secondary and tertiary amplifiers, create opportunities to use less sensitive detecting equipment and/or lower concentrations of the target material.

Experts in the art will recognize other areas of applicability of the techniques of the present invention and will, using these teachings, apply the concepts to yet other analytes of interest. Accordingly, the invention should be limited only by the scope of the claims appended hereto.

What is claimed is:

1. A method of discriminating between binding and nonbinding molecular targets of a molecular probe, the method comprising the steps of:
providing a substrate, said substrate forming a surface and an evanescent optical field proximate said surface;
immobilizing a molecular probe on said surface;
exposing said molecular probe to a solution containing said molecular target;
selectively perturbing said evanescent optical field only if said molecular target has bound to said molecular probe;
observing said evanescent field; and
correlating perturbation of said evanescent field with binding of said molecular target to said molecular probe.

2. A method as set forth in claim 1, wherein:
said substrate utilizes total internal reflection to form said evanescent field;
said molecular probe comprises a nucleic acid having a single-stranded segment;
said molecular target comprises a nucleic acid having a single-stranded segment tending to hybridize with said single-stranded segment of said molecular probe and thereby to form a double-stranded segment; and
said step of selectively perturbing said evanescent optical field includes the step of exposing said surface to a solution containing an immunoglobulin tending selectively to bind to said double-stranded segment.

3. A method as set forth in claim 2, wherein said step of selectively perturbing said evanescent optical field further includes the step of exposing said surface to a solution containing a material tending selectively to bind to said immunoglobulin.

4. A method as set forth in claim 1, wherein:
said substrate utilizes total internal reflection to form said evanescent field;
said molecular probe comprises an immunoglobulin; and
said molecular target comprises an antigen tending to bind to said immunoglobulin and thereby to form a complex.

5. A method as set forth in claim 4, wherein said step of selectively perturbing said evanescent optical field further includes the step of exposing said surface to a solution containing a material tending selectively to bind to said complex.

6. A method as set forth in claim 1, wherein:
said substrate utilizes total internal reflection to form said evanescent field;
said molecular probe comprises a nucleic acid having a single-stranded segment;
said molecular target comprises a nucleic acid having a single-stranded segment tending to hybridize with said single-stranded segment of said molecular probe and thereby to form a double-stranded segment; and
said step of selectively perturbing said evanescent optical field includes the step of exposing said surface to a mixture containing a mass-amplifying component, said mass-amplifying component comprising a sequence-specific polyamide tending selectively to bind to said double-stranded segment.

7. A method as set forth in claim 6, wherein said sequence-specific polyamide is bound to a member of the group consisting of: biotin, metallic microspheres, metallic colloid, polystyrene microspheres, nanospheres, avidin, streptavidin, and immunoglobulin.

8. A method as set forth in claim 2 wherein said immunoglobulin is mouse IgM tending selectively to bind to double-stranded DNA.

9. A method of discriminating between binding and nonbinding molecular targets of a molecular probe, the method comprising the steps of:
providing a substrate, said substrate forming a surface;
immobilizing a molecular probe on said surface;
exposing said molecular probe to a solution containing said molecular target;
selectively aggregating at least one high-molecular-weight component on said surface only if said molecular target has bound to said molecular probe;
detecting said high-molecular-weight component on said surface; and
correlating detection of said high-molecular-weight component with binding of said molecular target to said molecular probe.

10. A method as set forth in claim 9, wherein:
said comprises a planar slide;
said molecular probe comprises a nucleic acid having a single-stranded segment;
said molecular target comprises a nucleic acid having a single-stranded segment tending to hybridize with said single-stranded segment of said molecular probe and thereby to form a double-stranded segment; and
selectively aggregating at least one high-molecular-weight component on said surface includes the step of exposing said surface to a solution containing an immunoglobulin tending selectively to bind to said double-stranded segment.

11. A method as set forth in claim 10, wherein said step of selectively aggregating at least one high-molecular-weight component on said surface further includes the step of exposing said surface to a solution containing a material tending selectively to bind to said immunoglobulin.

12. A method as set forth in claim 9, wherein:
said comprises a planar slide;
said molecular probe comprises an immunoglobulin; and
said molecular target comprises an antigen tending to bind to said immunoglobulin and thereby to form a complex.

13. A method as set forth in claim 12, wherein said step of selectively aggregating at least one high-molecular-weight component on said surface further includes the step of exposing said surface to a solution containing a material tending selectively to bind to said complex.

14. A method as set forth in claim 9, wherein:
said comprises a planar slide;
said molecular probe comprises a nucleic acid having a single-stranded segment;
said molecular target comprises a nucleic acid having a single-stranded segment tending to hybridize with said single-stranded segment of said molecular probe and thereby to form a double-stranded segment; and
said step of selectively aggregating at least one high-molecular-weight component on said surface includes the step of exposing said surface to a mixture containing a mass-amplifying component, said mass-amplifying component comprising a sequence-specific polyamide tending selectively to bind to said double-stranded segment.

15. A method as set forth in claim 14, wherein said sequence-specific polyamide is bound to a member of the group consisting of: polyethylene glycol, biotin, metallic microspheres, metallic colloid, polystyrene microspheres, nanospheres, avidin, streptavidin, and immunoglobulin.

16. A method as set forth in claim 10 wherein said immunoglobulin is mouse IgM tending selectively to bind to double-stranded DNA.

17. A method for identifying an active substance in a sample by the reaction of the active substance (target) with its specific binding partner (probe) comprising the steps of:
a. Applying a sample probe material to a substrate;
b. Exposing said probe material to a target material; and
c. Applying a mass amplifier material which specifically binds to combinations of said probe and target materials,
whereby the resultant product of the probe-target combination is a binding site for said mass amplifier material providing a substantially more massive structure at the probe-target combination site, more easily recognized by detecting equipment.

18. The method of claim 17 wherein said probe material is an oligonucleotide and said target material is an oligonucleotide complementary with said probe material.

19. The method of claim 18 wherein said mass amplifier material is anti-double stranded DNA mouse IgM.

20. The method of claim 19 wherein said mass amplifier material is bonded to microspheres.

21. The method of claim 18, including the additional step of adding a dsDNA-sequence-specific minor-groove-binding molecule bonded to biotin prior to said step of adding a mass amplifier material.

22. The method of claim 17, including the additional step of applying a secondary amplifier material to target-probe-amplifier combination to provide a more massive combination.

23. The method of claim 19, including the additional step of applying, as a secondary amplifier material, goat anti-mouse IgM IgG.

24. The method of claim 21, including the additional step of applying, as a mass amplifier material, streptavidin.

25. The method of claim 17 wherein said probe material is non-human IgG and said target material is human IgG and said mass amplifier material is non-human anti-human-IgG IgG.

26. The method of claim 17 wherein said probe material is non-human IgG and said target material is human IgG and said mass amplifier material is non-human anti-human-IgG IgM.

27. A bioassay method for identifying a bioactive substance (antigen) comprising the steps of:
a. Fixing a probe (antibody) of the bioactive substance (antigen) to a substrate;
b. Applying the bioactive substance (antigen) to the substrate at the site of said probe (antibody) to form a reaction complex of said antigen and said antibody; and
c. Applying an amplifying complex (primary amplifying antibody) of a bulk and mass substantially greater than either said bioactive substance (antigen) or said probe (antibody) with an affinity for the said reaction complex of said antigen and said antibody;
whereby the reaction complex of said bioactive substance and said probe provides a binding site for said amplifying complex to provide an aggregate structure of mass and bulk substantially greater than the mass and bulk of the reaction complex.

28. The process of claim 27, further including the step of applying a second amplifying complex (secondary amplifying antibody) of a bulk and mass substantially greater than either said bioactive substance (antigen) or said amplifying complex (primary amplifying antibody); said second amplifying complex (secondary amplifying antibody) binding to said reacted combination and said complex (primary amplifying antibody) to further increase the mass and bulk at the site of the reaction complex.

29. Apparatus for identifying unknown target materials comprising;
a. A carrier having a first surface;
b. A plurality of probes each specific for a particular target material arranged in an identifiable subset; and
c. A quantity of mass enhancing amplifier material for forming probe-target-mass-enhancing-amplifier complexes at the site of a probe-target interaction,
whereby the presence of a probe-target interaction at a particular probe site is indicative of the identity of the target material.

30. Apparatus for identifying an unknown target material comprising in combination:
a. A carrier having a planar surface;
b. An array of probes immobilized on said surface, each of said probes exhibiting a known reaction with a different target; and
c. A mass enhancing amplifier material having a known unique attraction to combinations of said probes and respective corresponding targets,
whereby an unknown target material reacting with one of said array of probes binds to said mass enhancing amplifier material to create a readily distinguishable mass enhanced reaction product at the reaction site, thus uniquely identifying the unknown target material by the location of the reaction site within said array.

31. The apparatus of claim 30, wherein said carrier is a glass slide.

32. The apparatus of claim 30, further including an optical reader including transparent means having a first index of refraction and forming a total internal reflection from a surface thereof, wherein sad carrier has a second surface juxtaposed with said total internal reflection surface.

33. The apparatus of claim 30, wherein said carrier has an index of refraction chosen to be identical to that of said optical reader.

34. The apparatus of claim 30, wherein said probe is chosen from a class including oligonucleotides, single stranded DNA (ssDNA), non-human IgG, polymers, DNA-RNA hybrids, locked nucleic acids, polysaccharides, and proteins; wherein said targets are chosen from a class of materials that have a unique affinity for said probes; and wherein said mass-enhancing mass amplifier materials are chosen from a class of materials that have a unique affinity for probe-target combinations, including anti-double-stranded DNA mouse IgM, anti-double-stranded DNA mouse IgM coupled to microspheres, sequence seeker polyamide coupled to biotin, sequence seeker coupled to polyethylene glycol, sequence seeker polyamide coupled to anti-double stranded DNA mouse IgM, sequence seeker polyamide coupled to microspheres, anti-DNA-RNA hybrid mouse IgG, non-human anti-human-IgG IgG and non-human anti-human IgG, IgGM, the combination of probe and target in each instance being capable of producing a probe-target pair with a unique attraction for a mass-enhancing amplifier material selected from said class.

35. A method for identifying an unknown target material comprising the steps of:
a. immobilizing an array of probes, each specific for a different target material;
b. applying unknown target material to said array to form at least one probe-target pair; and
c. applying a mass-enhancing amplifier material that is adapted to bind to any formed probe-target pairs;
whereby any probe-target pair that is formed is more easily detected by the combination with the mass-enhancing amplifier material that attaches solely to probe-target pairs.

36. The method of claim 35, further including the step of applying a secondary mass enhancing amplifier material that is adapted to bond to formed probe-target pairs to which said mass enhancing amplifier material has attached to provide a significantly more massive probe-target site more readily identified by detection devices.

37. A bioassay method for identifying a bioactive substance (antibody) comprising the steps of:
a. Fixing a probe (antigen) of the bioactive substance (antibody) to a substrate;
b. Applying the bioactive substance (antibody) to the substrate at the site of said probe (antigen) to form a reaction complex of said antigen and said antibody; and
c. Applying an amplifying complex (primary amplifying antgen) of a bulk and mass substantially greater than either said bioactive substance (antibody) or said probe (antigen) with an affinity for the said reaction complex of said antigen and said antibody;
whereby the reaction complex of said bioactive substance and said probe provides a binding site for said amplifying complex to provide an aggregate structure of mass and bulk substantially greater than the mass and bulk of said reaction complex.

38. The process of claim 37, further including the step of applying a second amplifying complex (secondary amplifying antigen) of a bulk and mass substantially greater than either said bioactive substance (antibody) or said amplifying complex (primary amplifying antigen); said second amplifying complex (secondary amplifying antigen) binding to said reacted combination and said complex (primary amplifying antigen) to further increase the mass and bulk at the site of said reaction complex.

39. A method as set forth in claim 22, including the additional step of applying, as a tertiary amplifier material, an immunoglobulin specific to the secondary amplifier material.

40. A method as set forth in claim 39, wherein the tertiary amplifier material comprises IgM.

41. A method as set forth in claim 24, including the additional step of applying, as a tertiary amplifier material, a molecule having an affinity for streptavidin.

42. Apparatus comprising:
a. a slide having a first surface;
b. an array of DNA Segments (oligomers) immobilized on said surface;
c. matching DNA segments coupled to said oligomers for forming double-stranded hybridized complexes there; and
d. IgM molecules attached to said hybridized complexes.

43. Apparatus as in claim 42, wherein said IgM molecule is a fluorescent-labeled molecule.

44. Apparatus as in claim 42, wherein said IgM molecule is a magnetically-labeled molecule.

45. Apparatus as in claim 42, wherein said IgM molecule is a color-labeled molecule.

46. Apparatus as in claim 42, wherein said IgM molecule is labeled with high electric-permeability molecules.

47. Apparatus as in claim 42, wherein said array includes subarrays, each of said subarrays having immobilized there different DNA oligomers, different DNA matching segments coupled to the oligomers of at least one of said subarrays for forming double-stranded hybridized complexes there, said apparatus also including IgM molecules attached only to the hybridized complexes of at least said one of said subarrays.

48. The apparatus of claim 43, wherein said array includes subarrays, each of said subarrays having immobilized there different DNA oligomers, different DNA matching segments coupled to the oligomers of at least one of said subarrays for forming double-stranded hybridized complexes there, said apparatus also including IgM molecules attached only to the hybridized complexes of at least said one of said subarrays.

49. The apparatus of claim 44, wherein said array includes subarrays, each of said subarrays having immobilized there different DNA oligomers, different DNA matching segments coupled to the oligomers of at least one of said subarrays for forming double-stranded hybridized complexes there, said apparatus also including IgM molecules attached only to the hybridized complexes of at least said one of said subarrays.

50. The apparatus of claim 45, wherein said array includes subarrays, each of said subarrays having immobilized there different DNA oligomers, different DNA matching segments coupled to the oligomers of at least one of said subarrays for forming double-stranded hybridized complexes there, said apparatus also including IgM molecules attached only to the hybridized complexes of at least said one of said subarrays.

51. The apparatus of claim 46, wherein said array includes subarrays, each of said subarrays having immobilized there different DNA oligomers, different DNA matching segments coupled to the oligomers of at least one of said subarrays for forming double-stranded hybridized complexes there, said apparatus also including IgM molecules attached only to the hybridized complexes of at least said one of said subarrays.

52. A method for identifying an active substance in a sample by the reaction of the active substance (target) with its specific binding partner (probe) wherein the probe is a DNA segment (oligomer) and the target is a matching DNA segment capable of forming a hybridized (double-strand) complex with the probe, the method comprising the steps of:
a. immobilizing DNA segments (probes) on a carrier;
b. exposing said probes to applied targets for forming hybridized complexes; and
c. applying IgM molecules chosen for their ability to attach only to hybridized complexes.

53. A method as in claim 52, wherein said IgM is fluorescent-labeled.

54. A method as in claim 52, wherein said IgM is a magnetically-labeled molecule.

55. A method as in claim 52, wherein said IgM is a color-labeled molecule.

56. A method as in claim 52, wherein said IgM is labeled with high electric-permeability molecules.

57. A method as set forth in claim 1, wherein said molecular probe comprises a locked nucleic acid.

58. A method as set forth in claim 1, wherein:
said substrate utilizes total internal reflection to form said evanescent field;
said molecular probe comprises a nucleic acid having a single-stranded segment;
said molecular target comprises a nucleic acid having a single-stranded segment tending to hybridize with said single-stranded segment of said molecular probe and thereby to form a double-stranded segment; and
said step of selectively perturbing said evanescent optical field includes the step of exposing said surface to a solution containing a molecule tending selectively to bind to said double-stranded segment.

59. A method as set forth in claim 58, wherein said molecule tending selectively to bind to said double-stranded segment comprises a molecule selected from the group consisting of:
dsDNA-specific IgG,
dsDNA-specific Fab fragment,
dsDNA-specific F(ab′)2 fragment,
DNA-RNA-hybrid-specific IgG,
DNA-RNA-hybrid-specific Fab fragment,
DNA-RNA-hybrid-specific F(ab′)2 fragment,
DNA-LNA-hybrid-specific IgG,
DNA-LNA-hybrid-specific Fab fragment,
DNA-LNA-hybrid-specific F(ab′)2 fragment,
dsDNA-specific IgM tetramer,
dsDNA-specific IgM monomer,
dsDNA-specific IgM Fab fragment, and
dsDNA-specific IgM F(ab′)2 fragment.

60. A method as set forth in claim 1, wherein:
said substrate utilizes total internal reflection to form said evanescent field;
said molecular probe comprises a polysaccharice; and
said molecular target comprises an immunoglobulin tending to bind to said molecular probe and thereby to form a complex.

61. A method as set forth in claim 60, wherein said step of selectively perturbing said evanescent optical field further includes the step of exposing said surface to a solution containing a material tending selectively to bind to said complex.
